(19) United States
(12) Patent Application Publication
(10) Pub. No.: US 2014/0076990 A1
(43) Pub. Date: Mar. 20, 2014

(54) PRINTED COMPOSITE FOR DIFFUSING FRAGRANCES AND MANUFACTURING METHOD AND DEVICE

(71) Applicant: PO-DREUX, Dreux Cédex (FR)

(72) Inventors: Carole Lopandia, La Madeleine De Nonancourt (FR); Benoît Frassaint, Mont Saint Aigman (FR)

(73) Assignee: PO-DREUX, DREUX Cedex (FR)

(21) Appl. No.: 14/118,676

(22) PCT Filed: May 10, 2012
(86) PCT No.: PCT/EP2012/058681

§ 371 Date: Dec. 3, 2013

(30) Foreign Application Priority Data
May 20, 2011 (FR) 1154429

(51) Int. Cl. A61L 9/12

(52) U.S. Cl. CPC A61L 9/12
USPC 239/53; 156/226; 156/390; 264/134; 425/90

(57) ABSTRACT

The present invention relates to a printed composite for distributing a fragrance (4), characterized in that the composite includes: a printed and/or printable absorbent substrate-reservoir layer (1), which is suitable for accommodating a fragrance (4) that is intended to be diffused; and a printed internal barrier layer (2) that is positioned against one surface (1B) of the printed and/or printable absorbent substrate-reservoir layer (1) in order to control the migration and/or diffusion of the fragrance (4).

### DESCRIPTION

The present invention relates to the field of diffusers diffusing odoriferous substances and more particularly to the field of diffusers allowing the controlled, sustained diffusion of the odoriferous substances they contain.

It is becoming increasingly more frequent to offer samples of a perfume in the form of an impregnated blotter attached to an advertising insert in a magazine. Additionally, these samples are frequently offered in the form of a blotter such as a fragrance card or impregnated card given directly to potential clients in perfume stores. Similarly, to set themselves aside from competitors, some trade names use an olfactory signature on their business cards or in mail or on publicity items intended for the launch of new sales campaigns.

One major disadvantage of these impregnated blotters is that their capacity to deliver the odoriferous substance leads to non-desired and non controlled diffusion when they come into contact with an item in absorbent material. Therefore these blotters, when intended to be arranged between the pages of magazines, lose some of their odoriferous substance due to a phenomenon of migration in the pages between which they are positioned. Over and above the first problem of loss of a certain amount of the deposited odoriferous substance, there is the added problem relating firstly to deterioration of the odoriferous substance by the inks of the magazine pages which may react with the composition stored in the blotter, and secondly to soiling of the magazine pages between which the blotters are arranged.

U.S. Pat. No. 5,093,182 proposes an example of this application of a perfume on a printed advertising substrate. This particular application is designed so that the fragrance deposited on the advertising substrate does not interact with the ink of the printed surface. To achieve this, the oil of the fragrance is previously mixed, in the absence of any aqueous phase, with a polymer and in particular with ethyl cellulose which is a film-forming agent known to allow controlled viscosity. The fragrance/polymer mixture is then suitable for depositing on a printed substrate via a known offset system. Once deposited this fragrance has the characteristic of being quick-drying without requiring any heating. However said solution is not satisfactory insofar as the migration and/or diffusion of the deposited fragrance are not controlled within the printed substrate or between several printed substrates.

Patent application JP 9076377 also proposes a method for fixing a fragrance onto a sampler. According to this method a substrate receives an odoriferous fragrance and this odoriferous substance is then coated with a membrane sealed onto the substrate. This membrane prevents early evaporation or diffusion of the deposited fragrance when the substrate is stored. The release of the odoriferous substances from the substrate is obtained by a user removing the membrane from the surface of the substrate. However, said method does not propose a solution which allows the direct depositing of an odoriferous substance onto a printed substrate. In addition, while storing of the product allows preserving of the odoriferous substance, the release of this substance remains dependent on removal of the membrane.

Patent JP 2003063174 proposes a solution enabling a substrate to contain two perfumes by means of a pair of sheets joined along a fold line. Each of these sheets, along its outer edge, has a strip coated with a respective perfume and which is folded and glued against a surface of the sheet forming a pocket or envelope inside which the perfume is positioned, so as to avoid mixing of the perfumes when the sheets each containing a different perfume are folded over one another. This technique could be used to limit the problems of undue absorption by magazine pages. But by preventing this absorption this technique also limits diffusion since it requires the user to open and tear the pocket or envelope to release the odoriferous substance contained therein. In addition, once the pocket is opened it can no longer be closed and the magazine pages may become soiled.

Patent application US 2002/0013108 describes a blotter having a first layer in paper or plastic material having a printed outer surface, and a highly porous second layer fixed to the inner surface of the first layer by gluing or pressure and impregnated with perfume so as to diffuse a perfume by rubbing against the skin. This solution is not satisfactory since the perfume may freely migrate between the different layers thereby creating various technical disorders, and no provision is made for printing on the blotter layer.

U.S. Pat. No. 6,125,614 presents a laminated page comprising a carrier sheet of paper folded and sealed to contain a sampler material positioned in this fold. More specifically, a barrier layer in material impermeable to the sampler material e.g. PET polymer is fixed to the paper carrier sheet by an adhesive and folded together with it, thereby providing a first lower barrier portion and second upper barrier portion between which the sampler material is arranged. This laminated page does not allow the easy release of the sampler material and requires detaching of the fold.

The present invention sets out to overcome one or more disadvantages of the prior art and in particular to propose a solution with which it is possible to control the diffusion of an odoriferous substance by controlling its migration, whilst preventing degradation of its odoriferous characteristics by the substrate materials used or by the printing processes applied.

The present invention also proposes solving the problems of industrial production and the control of quality throughout the production line and during storage and distribution of the product.

This objective is reached by means of a printed composite for the diffusion of an odoriferous substance, characterized in that the composite comprises:
an absorbent layer of printable and/or printed reservoir-substrate adapted to receive an odoriferous substance intended to be diffused;
a printed inner barrier layer arranged against one surface of the absorbent layer of printable and/or printed reservoir-substrate, to control the migration and/or diffusion of the odoriferous substance.

According to one particular aspect of the invention, the printed composite is characterized in that the absorbent layer of the printable and/or printed reservoir-substrate is in cellulose fibres.

According to another particular aspect of the invention, the printed composite is characterized in that a first surface of the absorbent layer of printable and/or printed reservoir-substrate, intended to receive the odoriferous substance, is macroporous of uncoated type.

According to another particular aspect of the invention, the multilayer composite is characterized in that a second surface of the absorbent layer of printable and/or printed reservoir-substrate is preferably of coated type.

According to another particular aspect of the invention, the printed composite is characterized in that the printed inner barrier layer is a polymer film or varnish-type coating.

According to a further particular aspect of the invention, the printed composite is characterized in that the inner barrier layer arranged against the absorbent layer of printable and/or printed reservoir-substrate forms a first so-called inner migration barrier, the printed composite also comprising an outer barrier layer arranged on the printed surface of the inner barrier layer forming a second so-called outer migration barrier.

According to a further particular aspect of the invention, the printed composite is characterized in that the outer barrier layer is a polymer film or varnish-type coating.

According to a further particular aspect of the invention, the printed composite is characterized in that the odoriferous substance deposited on the first surface of the absorbent layer of printable and/or printed reservoir-substrate is composed of at least one essential oil.

According to a further particular aspect of the invention, the printed composite is characterized in that the composite also comprises at least one hinge axis to allow the overlapping of a first portion of the first surface of the absorbent layer of printable and/or printed reservoir-substrate, which is not in contact with the inner barrier layer, over a second portion of this same surface.

A further object of the invention is to propose a method allowing the production of the printed composite of the invention.

This object is achieved by means of a method for manufacturing a printed composite for diffusion of an odoriferous substance according to the invention, characterized in that the method particularly comprises:
a step to fix an inner barrier layer, such as a polymer film or varnish coating, on the second surface of the absorbent layer of printable and/or printed reservoir-substrate which is not intended to receive the odoriferous substance, so as to form a complex of absorbent layer of printable and/or printed reservoir-substrate/inner barrier layer,
a step to deposit an odoriferous substance on the first surface of the absorbent layer of reservoir-substrate.

According to one particular aspect of the invention, the manufacturing method is characterized in that the method also comprises a step to fix an outer barrier layer on the printed surface of the inner barrier layer.

According to a further particular aspect of the invention, the manufacturing method is characterized in that the step to deposit an odoriferous substance is performed by distributing the substance over a defined surface of the absorbent layer of printable and/or printed reservoir-substrate.

According to a further particular aspect of the invention, the method also comprises a step to fold the printed composite so that a first portion of the first surface of the absorbent layer of reservoir-substrate, which is not in contact with the inner barrier layer, overlaps a second portion of this same surface.

A further object of the invention is to propose a system allowing the implementation of the method to manufacture the printed composite of the invention.

This object is achieved by means of a system for manufacturing a printed composite for diffusion of an odoriferous substance, characterized in that it is adapted for implementing a manufacturing method according to the invention, the system notably comprising:
a station to fix an inner barrier layer, such as a polymer film or varnish coating, onto the second surface of the absorbent layer of printable and/or printed reservoir-substrate which is not intended to receive the odoriferous substance, so as to form a complex of absorbent layer of printable and/or printed reservoir-substrate/inner barrier layer,
a station to deposit an odoriferous substance on the first surface of the absorbent layer of reservoir-substrate.

The invention with its characteristics and advantages will become better apparent on reading the description given with reference to the appended drawings in which:

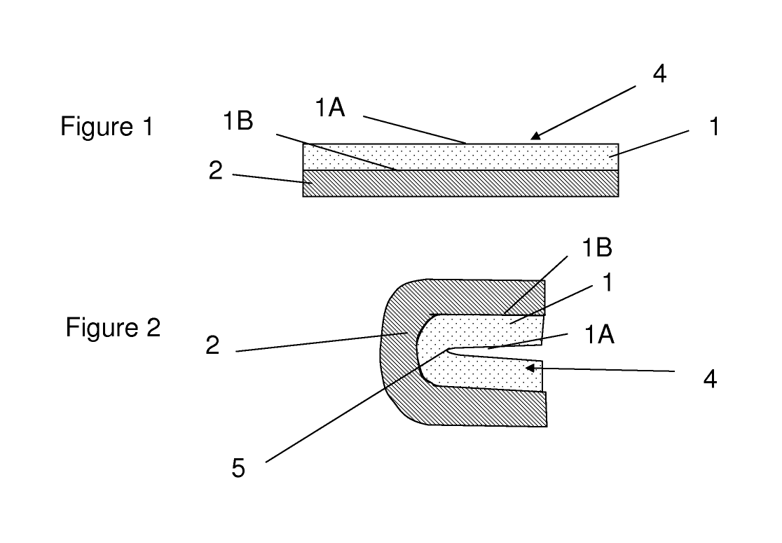

FIG. 1 schematically illustrates the superimposed bi-layer (absorbent layer of reservoir-substrate/inner barrier layer) allowing the receiving of an odoriferous substance in the absorbent layer of reservoir-substrate;

FIG. 2 schematically illustrates a variant of the bi-layer of the invention wherein it is folded to prevent the diffusion of the odoriferous substance;

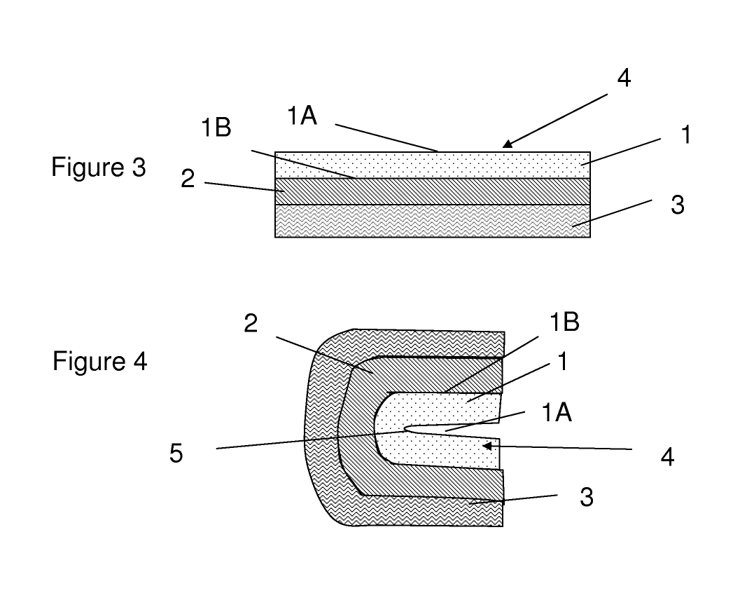

FIG. 3 schematically illustrates a variant of the printed composite in which an outer barrier layer is fixed onto the printed surface of the inner barrier layer;

FIG. 4 schematically illustrates a variant of the printed composite of the invention in which it is folded to prevent diffusion of the odoriferous substance;

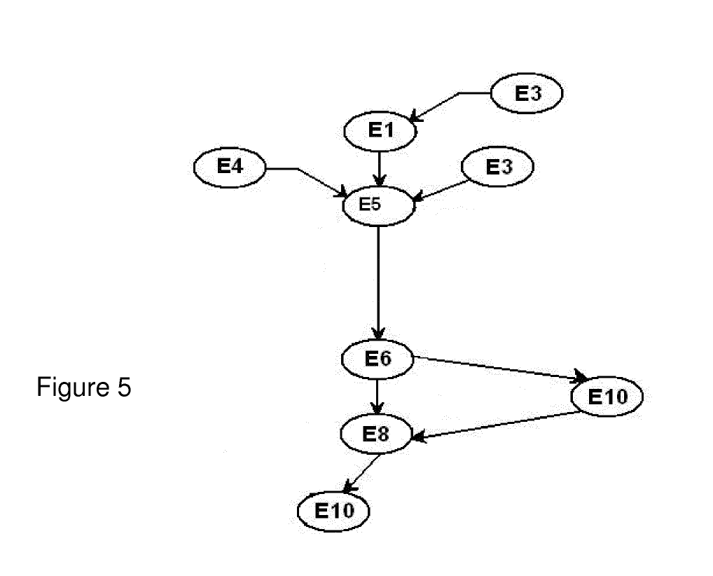

FIG. 5 schematically illustrates the different possible steps of a method to manufacture the composite of the invention;

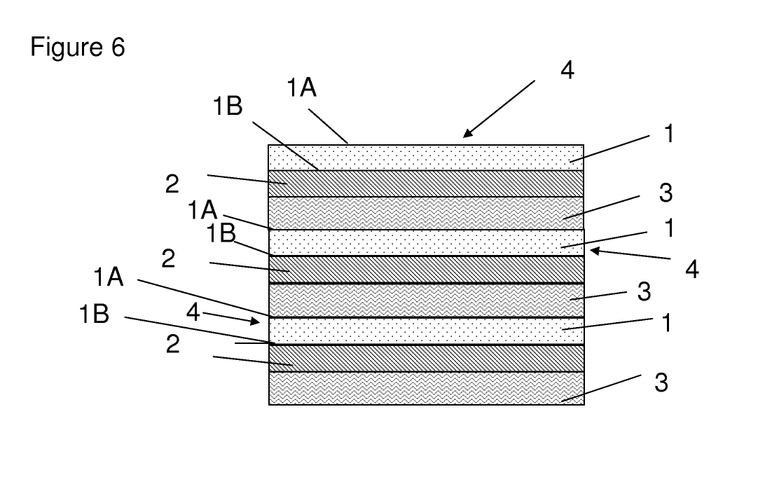

FIG. 6 shows a stack of printed composites during the transport and/or storage thereof before folding;

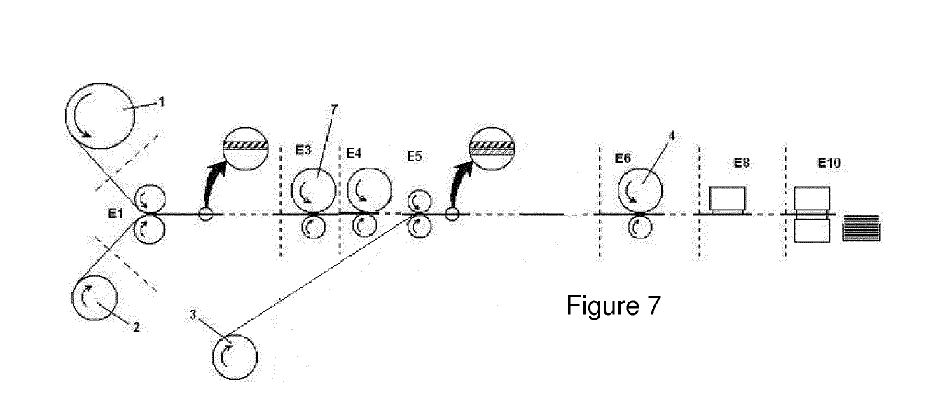

FIG. 7 schematically illustrates a device allowing the implementation of the manufacturing method of the invention.

The composite of the invention is formed by a superimposed composite assembly arranged to receive and store or trap an odoriferous substance 4 during storage and at the different steps of industrial production or at the time of distribution of the products, and to allow the diffusion thereof when the fold-out is opened by the end user (reader of a magazine or mail, shop customer).

This printed composite assembly is particularly adapted, as an illustrative but non-limiting example, for the perfuming of printed substrates used in numerous industries and in particular the packaging and cosmetic industries (manufacturers of sampler cases, boxes and cards) POS manufacturers (Point-of-sale advertising) and printing industries (press inserts, mailed or non-mailed advertising, flyers, olfactory marketing).

In particular, it is suitable for producing advertising inserts or sampler cards to be inserted in the pages of a journal, a magazine and to diffuse fragrances associated with the communication product illustrated or printed on the insert or sampler card.

The odoriferous substance 4 is volatile and may be a fragrance, an essential oil, alone or combined with a solvent, an aromatic compound, a diluent, a polymer, even a resin.

The odoriferous substance 4 is formulated in liquid or paste phase of oil, cream or gel type, having adapted viscosity so that the odoriferous substance 4 can be deposited in or on the absorbent layer of printable and/or printed reservoir-substrate 1.

The absorbent layer is thereby impregnated with the odoriferous substance via absorption of the odoriferous substance

According to one preferred embodiment, this odoriferous substance 4 coats at least one part of the surface of one of the layers located on the outside 1A of the composite assembly, through the depositing of a predefined quantity. This deposit can be performed by coating all or part of the surface of the outer layer 1A of the composite.

Preferably, this odoriferous substance 4 is deposited in liquid phase.

According to one example of embodiment, the odoriferous substance 4 can be integrated by moistening the absorbent layer of reservoir substrate 1 thereby allowing the storage and diffusion of the odoriferous substance 4.

Preferably the absorbent layer of reservoir-substrate 1 is made in paper or paperboard of gram weight equal to or higher than 200 g/m².

This paper or paperboard is in pure cellulose fibres and is formed of a first uncoated surface 1A of macroporous type so that the odoriferous substance 4 is able to be absorbed by the fibres.

The uncoated surface 1A of paperboard is particularly suitable for printing.

In this case, printing inks should be used that are compatible with the odoriferous substance 4 so that these do not interact or deteriorate the olfactory characteristics of the odoriferous substance 4. According to one preferred but non-limiting embodiment of the invention, the printing inks used are of UV type and are set by photo-polymerisation when in contact with UV radiation (Ultraviolet). The inks can also be dried by IR radiation (infrared).

Additionally, this paper or paperboard in cellulose fibres is formed of a second preferably coated surface 1B of microporous type adapted to receive and ensure the proper fixing of the inner barrier layer 2 and to impart a tensioned appearance and surface condition.

This may be paperboard composed of pure cellulose fibres adapted so as not to interact via oxidation in contact with the odoriferous substance 4.

In noteworthy manner, with this paper or paperboard and on account of the intermingling of the cellulose fibres, the diffusion of the odoriferous substance 4 may be in the order of a few days to several weeks in open air depending on the deposited amount and depending on the formulation of the odoriferous substance 4.

As illustrated in FIG. 1 the absorbent layer of reservoir-substrate 1 is fixed via its second surface 1B to an inner barrier layer 2 formed for example of a polymer film or varnish coating, which ensures a barrier against migration of the odoriferous substance 4 towards the outside of the surface 1B.

According to a first example of embodiment, the inner barrier layer 2 is formed of a polymer film of PET or treated OPP type. Thus formed, this inner barrier layer 2 can prevent any migration of the odoriferous substance 4 towards the outside of the surface 1B.

As a non-limiting example, if the inner barrier layer 2 is a film of treated OPP it may have a thickness of between 12 and 20 μm and a gram weight of between 11 and 18 g/m². The treatment of the polypropylene film can be obtained by plasma treatment or chemical treatment which allows a surface energy to be obtained of more than 38 dynes whereby the treated OPP film becomes printable.

As a non-limiting example, if the inner barrier layer 2 is a PET film, it may have a thickness of 12 μm and gram weight of about 17 g/m². The surface condition of the PET film has a surface energy of more than 38 dynes allowing the printing thereof.

According to a second example of embodiment, the inner barrier layer 2 is formed by the application of a varnish coating. Thus deposited, this inner barrier layer 2 allows the preventing of any migration of odoriferous substance 4 towards the outside of the second surface 1B.

As non-limiting example, the inner barrier layer 2 is a UV varnish deposited by flexography and having a gram weight of 4 to 6 g/m². This varnish is particularly formulated to obtain a surface energy higher than 38 dynes and to allow printability of the inner barrier layer 2.

In this case, printing inks should be used that are compatible with the fixed inner barrier layer 2.

According to one preferred but non-limiting embodiment of the invention, the printing inks used are of UV type and are set by photo-polymerisation when placed in contact with UV radiation (Ultraviolet). Ink drying can also be obtained using IR radiation (Infrared).

According to one embodiment of the invention illustrated in FIG. 3, the printed composite for diffusion of an odoriferous substance also comprises an outer barrier layer 3 on the surface of the inner barrier layer 2 which is not in contact with the absorbent layer of reservoir-substrate 1B.

According to one particular embodiment which does not limit the invention, the outer barrier layer 3 is formed for example by a polymer film or varnish coating.

Advantageously this outer barrier layer 3 allows the preventing of migration and diffusion of odoriferous substances 4 from the absorbent layer of printable and/or printed reservoir-substrate 1 of a first printed composite arranged underneath a second printed composite, in particular during stacking or storage or when transporting a plurality of printed composites of the invention as illustrated in FIG. 6. The two least sheets of printed composite located on the top of the stack can be arranged head-to-tail i.e. in contact with one another at their first outer surface 1A which prevents or limits the diffusion of the odoriferous substance 4 owing to the imperviousness of the outer barrier layer 3 located at the end of the stack.

This outer barrier layer 3 for example, depending on its characteristics, may have a surface of printed composite of the invention that can be glued onto a substrate. This outer barrier layer 3 can therefore be glued and allows fixing of the printed composited onto a substrate such as, but not limited thereto, a magazine or advertising item or a display item of POS type (Point-Of-Sale).

In one alternative embodiment illustrated in FIG. 4, the printed composite of the invention also comprises at least one hinge axis 5 to allow the overlapping of a first portion of the outer surface 1A of the absorbent layer of reservoir-substrate 1, which is not in contact with the inner barrier layer 2, over a second portion of this same outer surface 1A. All the layers of the bi-layer (absorbent layer of reservoir-substrate 1, inner barrier layer 2) and of the triple layer (absorbent layer of reservoir-substrate 1, inner barrier layer 2, outer barrier layer 3) are jointly folded as illustrated in FIGS. 2 and 4.

This hinge axis 5 can be formed for example by scoring or axial folding. Therefore when the absorbent layer of printable and/or printed reservoir-substrate 1 comprises an odoriferous substance 4, the overlapping obtained by folding the two portions of the surface of absorbent layer of reservoir-substrate 1 one over the other towards a closed position allows the limiting even preventing of diffusion of the odoriferous substance 4.

The release by diffusion of the odoriferous substance 4 is then obtained by a user who opens and/or unfolds the printed composite of the invention (opening of the magazine, opening of the sampler card, opening of a direct marketing fold-out . . . ).

Therefore the printed composite of the invention allows a substrate to be formed for the diffusion of an odoriferous substance 4 which is able to be associated with a printed visual communication item (text, trade name, advertising message, etc.) both on the part formed by the absorbent layer of reservoir-substrate 1 and on the part formed by the inner barrier layer 2.

The manufacture of this printed composite of the invention requires several successive steps in a manufacturing method illustrated in FIGS. 5 and 7, the order of which may differ for some thereof.

Therefore the method for manufacturing a printed composite of the invention particularly comprises:
a step (E1) to fix an inner barrier layer 2 such as a polymer film or varnish coating on the second surface 1B of the absorbent layer of printable and/or printed reservoir-substrate 1 which is not intended to receive the odoriferous substance 4, so as to obtain an absorbent layer of printable and/or printed reservoir-substrate/inner barrier layer 2 complex;
a step (E6) to deposit an odoriferous substance 4 on the surface 1A of the absorbent layer of reservoir-substrate 1.
The step (E1) to fix the absorbent layer of reservoir-substrate 1 onto the inner barrier layer 2 is performed by forming a film and/or complexing and/or varnish coating.

The deposit step (E6) can be performed by coating, printing the surface of the absorbent layer of reservoir-substrate 1 with the odoriferous substance 4 intended to be diffused. This deposit step can use serigraphy, flexography or offset printing techniques. These techniques allow distribution of the odoriferous substance 4 over all or part of the surface of the absorbent layer of reservoir-substrate 1 and allow a variation in the quantity of deposited odoriferous substance 4.

During the manufacture of the printed composite, as illustrated in FIGS. 3 and 5, it may be necessary to performing printing (E3) on the surface 1A of the absorbent layer of reservoir-substrate 1 using an appropriate printing device 7. This printing (E3) can be performed either prior to the fixing step (E1) to fix the inner barrier layer 2 on the surface of the absorbent layer of reservoir-substrate 1, or after this step, but preferably prior to the deposit step (E6) to deposit the odoriferous substance 4 on the absorbent layer of reservoir-substrate 1.

Similarly, the method comprises a printing step (E4) of the inner barrier layer 2 with an appropriate printing device and/or system. This printing step (E4) is performed after the fixing step (E1) to fix the absorbent layer of reservoir-substrate 1 on the inner barrier layer 2. This printing step (E4) is conducted prior to deposit step (E6) to deposit the odoriferous substance 4 on the absorbent layer of reservoir-substrate 1.

Similarly, prior to the deposit step (E6) to deposit an odoriferous substance 4 on the absorbent layer of reservoir-substrate 1 the method may comprise a step (E5) to fix an outer barrier layer 3 on the printed barrier layer 2.

The printed composite being thus formed, and the odoriferous substance 4 optionally being deposited (E6), the method may further comprise a finishing step (E8). This finishing step (E8) for example may entail one or more operations from a list comprising for example gilding, embossing, cut-out, scoring . . .

According to a first particular embodiment which does not limit the principle of the invention, the homogeneity of distribution and the determination of the surface intended to be coated with the odoriferous substance 4 are defined by a screen of varying mesh size (similar principle to the printing technique of serigraphy type). As a non-limiting example, it is possible with this method to deposit a quantity of odoriferous substance of 12 to 20 g/m².

According to a second particular embodiment which does not limit the principle of the invention, the homogeneity of distribution may also have recourse to an anilox which is a cylinder having on its peripheral surface different forms of etchings or alveoli which, depending on their respective shapes and volumes and in combination with a blanket, contribute towards measurement of the amount of odoriferous substance 4 intended to be deposited on the absorbent layer of reservoir-substrate 1 (similar principle to the printing technique of flexography type). As a non-limiting example, it is possible with this method to deposit a quantity of odoriferous substance 4 of 3 to 10 g/m².

According to a third particular embodiment, a metal surface e.g. forming a plate is used to transfer the odoriferous substance 4 onto an elastomer surface forming a blanket for example. The elastomer surface is positioned in contact with the surface of the absorbent layer of reservoir-substrate 1 (similar principle to the printing technique of offset type). As a non-limiting example it is possible with this method to deposit a quantity of odoriferous substance 4 in the order of one or a few g/m².

The printed composite being formed and the odoriferous substance 4 being deposited, the system of the invention allows the transport and storage of the printed composite in the form of sheets or flat prints stacked so that the diffusion or migration of the odoriferous substance 4 are controlled.

According to one preferred embodiment, the method of the invention also comprises a folding step (E10) to fold the printed composite so that a first portion of the outer surface 1A of the absorbent layer of reservoir-substrate 1, which is not in contact with the inner barrier layer 2, overlaps a second portion of this same outer surface 1A of the absorbent layer of reservoir-substrate 1. Preferably this folding operation (E10) is performed after the deposit step (E6) of an odoriferous substance 4 on the printed composite. Optionally, the deposit step (E6) to deposit the odoriferous substance 4 on the absorbent layer of reservoir-substrate 1 can be performed after all the other manufacturing steps at a point of sale for the distribution or final display of the printed composite to the client.

In addition, the manufacturing system allowing the implementation of the method of the invention can be formed of a single device or of a plurality of separate devices. These devices then comprise one or more stations arranged to carry out at least one of the different operations or steps of the manufacturing method to manufacture the printed composite of the invention.

According to one particular embodiment which does not limit the invention and illustrated in FIG. 7, the system of the invention comprises a plurality of stations each being adapted to allow the performing of at least one of the steps: fixing of barrier layers onto the composite substrate (E1, E5), printing (E3, E4), depositing of odoriferous substance (E6) or finishing (E8) steps of the manufacturing method of the invention.

According to one preferred embodiment which does not limit the invention the device also comprises a central unit which allows the coordinated functioning of the different stations in the system. This coordination may simply concern two stations or all the stations of the system, the central unit being able to control at least one station of the device.

As already described previously FIG. 5, which gives a schematic illustration of the different steps of the manufacturing method of the printed composite of the invention, clearly shows that some steps of the method in particular the printing steps (E3, E4) can be performed at different times of the method compared with the main steps to form the bi-layer (E1) and to deposit the odoriferous substance (E6) of the composite.

The system for implementing the method of the invention comprises a plurality of stations each of which is organised, arranged or designed to carry out at least one step respectively of the method of the invention previously described. According to one non-limiting embodiment of the invention, the stations of the system for implementing the method of the invention may be positioned on one or several different production sites. The products obtained at each of these stations can then be transported from one site to the other for the continued implementation of the method. Similarly, the system of the invention may integrate a central unit which allows the management and coordination of the system for producing the composite.

It will be obvious for persons skilled in the art that the present invention allows embodiments under numerous other specific forms without departing from the field of application of the invention as claimed. Therefore the present embodiments are to be construed as illustrations but can be modified within the field defined by the scope of the appended claims.

1. A printed composite for diffusion of an odoriferous substance (4), wherein the composite comprises:
an absorbent layer of printable and/or printed reservoir-substrate (1) adapted to receive an odoriferous substance (4) intended to be diffused;
a printed inner barrier layer (2) arranged against one surface (1B) of the absorbent layer of printable and/or printed reservoir-substrate (1), to control the migration and/or diffusion of the odoriferous substance (4); and
at least one hinge point (5) to allow the overlapping of a first portion of the surface (1A) of the absorbent layer of printable and/or printed reservoir-substrate (1), which is not in contact with the inner barrier layer (2), over a second portion of this same surface (1A).

2. The printed composite according to claim 1, wherein the absorbent layer of printable and/or printed reservoir-substrate (1) is in cellulose fibres.

3. The printed composite according to claim 2, wherein a first surface (1A) of the absorbent layer of printable and/or printed reservoir-substrate (1) intended to receive the odoriferous substance (4) is macroporous, of uncoated type.

4. The printed composite according to claim 1, wherein a second surface (1B) of the absorbent layer of printable and/or printed reservoir-substrate (1) is preferably of coated type.

5. The printed composite according to claim 1, wherein the printed inner barrier layer (2) is a polymer film or varnish-type coating.

6. The printed composite according to claim 1, wherein the inner barrier layer (2) arranged against the absorbent layer of printable and/or printed reservoir-substrate (1) forms a first so-called inner barrier against migration, the printed composite also comprising an outer barrier layer (3) arranged on the printed surface of the inner barrier layer (2) forming a second so-called outer barrier against migration.

7. The printed composite according to claim 6, wherein the outer barrier layer (3) is a polymer film or varnish-type coating.

8. The printed composite according to claim 1, wherein the odoriferous substance (4) deposited on the first surface (1A) of the absorbent layer of printable and/or printed reservoir-substrate (1) is composed of at least one essential oil.

9. A method for manufacturing a printed composite for diffusion of an odoriferous substance according to claim 1, wherein the method particularly comprises:
a step to fix an inner barrier layer (2) such as a polymer film or varnish coating onto the second surface (1B) of the absorbent layer of printable and/or printed reservoir-substrate (1) which is not intended to receive the odoriferous substance (4), so as to obtain a complex of absorbent layer of printable and/or printed reservoir-substrate (1)/inner barrier layer (2);
a step to deposit an odoriferous substance (4) on the first surface (1A) of the absorbent layer of reservoir-substrate (1); and
a step to fold the printed composite so that a first portion of the first surface (1A) of the absorbent layer of reservoir-substrate (1), which is not in contact with the inner barrier layer (2), overlaps a second portion of this same surface (1A).

10. The manufacturing method according to claim 9, wherein the method also comprises a step to fix an outer barrier layer (3) onto the printed surface of the inner barrier layer (2).

11. The manufacturing method according to claim 9, wherein the step to deposit an odoriferous substance (4) is performed by distributing the substance over a defined surface of the absorbent layer of printable and/or printed reservoir-substrate (1).

12. A system for manufacturing a printed composite for diffusion of an odoriferous substance (4), wherein it is adapted for implementing a manufacturing method according to claim 9, the system notably comprising:
a station to fix an inner barrier layer (2) such as a polymer film or varnish coating onto the second surface (1B) of the absorbent layer of printable and/or printed reservoir-substrate (1) which is not intended to receive the odoriferous substance (4), so as to obtain a complex of absorbent layer of printable and/or printed reservoir-substrate (1)/inner barrier layer (2); and
a station to deposit an odoriferous substance (4) on the first surface (1A) of the absorbent layer of reservoir-substrate (1).
